(19) United States
(12) Patent Application Publication
(10) Pub. No.: US 2011/0160565 A1
(43) Pub. Date: Jun. 30, 2011

(54) DETECTING PROXIMITY TO MRI SCANNER

(76) Inventors: Scott R. Stubbs, Maple Grove, MN (US); Keith R. Maile, New Brighton, MN (US)

(21) Appl. No.: 12/956,034

(22) Filed: Nov. 30, 2010

Related U.S. Application Data
(60) Provisional application No. 61/291,435, filed on Dec. 31, 2009.

(51) Int. Cl. A61N 1/08; A61N 1/37; A61B 5/055

(52) U.S. Cl. 600/411; 607/62; 607/11

(57) ABSTRACT

A plurality of separate indications of a scanner field can be received using a corresponding plurality of scanner field sensors of an implantable medical device (IMD). In an example, the IMD can be switched from a first therapy mode to a second therapy mode using one or more of the plurality of scanner field sensors, and from the second therapy mode back to the first therapy mode using each of the plurality of scanner field sensors. In certain examples, shock therapy can be terminated or inhibited using a detected proximity of the IMD to a magnetic resonance imaging (MRI) scanner, or antitachycardia pacing (ATP) can be terminated or inhibited using a detected active scan of the MRI scanner.

### CLAIM OF PRIORITY

This application claims the benefit of priority under 35 U.S.C. §119(e) to Stubbs et al. U.S. Provisional Patent Application Ser. No. 61/291,435, entitled “DETECTING PROXIMITY TO MRI SCANNER,” filed on Dec. 31, 2009 (Attorney Docket No. 279.107PRV), which is hereby incorporated by reference herein in its entirety.

### BACKGROUND

Implantable medical devices (IMDs) can perform a variety of diagnostic or therapeutic functions. For example, an IMD can include one or more cardiac function management features, such as to monitor or to provide electrical stimulation to a heart or to the nervous system, such as to diagnose or treat a subject, such as one or more electrical or mechanical abnormalities of the heart. Examples of IMDs can include pacers, automatic implantable cardioverter-defibrillators (ICDs), or cardiac resynchronization therapy (CRT) devices, among others. Nuclear magnetic resonance imaging (MRI) is a medical imaging technique that can be used to visualize internal structure of the body. MRI is an increasingly common diagnostic tool, but can pose risks to a person with an IMD, such as a patient undergoing an MRI scan or a person nearby MRI equipment, or to people having a conductive implant.

### OVERVIEW

In an example, a plurality of separate indications of a scanner field (e.g., a magnetic field, an electric field, or a combination of a magnetic and electric field indicative of a nuclear magnetic resonance scan) can be received using a corresponding plurality of scanner field sensors (e.g., a magnetic field sensor, an electrical field sensor, combinations of each, etc.) of an implantable medical device (IMD). In an example, the IMD can be switched from a first therapy mode to a second therapy mode using one or more of the plurality of scanner field sensors, and from the second therapy mode back to the first therapy mode using each of the plurality of scanner field sensors. In certain examples, shock therapy can be terminated or inhibited using a detected proximity of the IMD to a magnetic resonance imaging (MRI) scanner, or antitachycardia pacing (ATP) can be terminated or inhibited using a detected active scan of the MRI scanner.

In Example 1, a system includes an implantable medical device (IMD) configured to be implanted into a subject, the IMD including a first therapy mode and a second therapy mode, the second therapy mode different than the first therapy mode, a plurality of scanner field sensors configured to receive separate indications of a scanner field, wherein the IMD is configured to receive each of the separate indications of the scanner field, to switch from the first therapy mode to the second therapy mode using one or more of the plurality of scanner field sensors, and to switch from the second therapy mode to the first therapy mode using each of the plurality of scanner field sensors.

In Example 2, the scanner field of Example 1 optionally includes a static or time-varying magnetic or electric field associated with a nuclear magnetic resonance (NMR) device.

In Example 3, the IMD of any one or more of Examples 1-2 is optionally configured to switch from the first therapy mode to the second therapy mode if any one or more of the plurality of scanner field sensors receives an indication of a scanner field.

In Example 4, the IMD of any one or more of Examples 1-3 is optionally configured to switch from the second therapy mode to the first therapy mode only if each of the plurality of scanner field sensors receives an indication of no scanner field.

In Example 5, the first therapy mode of any one or more of Examples 1-2 includes normal operation of the IMD, and wherein the second therapy mode includes at least one of a terminated or inhibited shock therapy or a terminated or inhibited antitachycardia pacing (ATP).

In Example 6, the plurality of scanner field sensors of any one or more of Examples 1-5 optionally includes at least one static magnetic or electric field sensor and at least one active magnetic or electric field sensor.

In Example 7, the plurality of scanner field sensors of any one or more of Examples 1-6 optionally includes a first scanner field sensor configured to detect an indication of a magnetic field, the first scanner field sensor including an inductor having a ferromagnetic core and a core saturation detector configured to detect saturation of the inductor and the ferromagnetic core in a strong magnetic field, a second scanner field sensor configured to detect an indication of a magnetic field, the second magnetic field sensor including a Hall effect sensor, and a third scanner field sensor configured to detect an indication of a magnetic or electric field, the third scanner field sensor including an electromagnetic interference (EMI) sensor configured to detect EMI from an active magnetic resonance imaging (MRI) scan.

In Example 8, at least one of the plurality of scanner field sensors of any one or more of Examples 1-7 are optionally configured to detect a proximity to an MRI scanner, and wherein the IMD is configured to terminate or inhibit shock therapy using the detected proximity to the MRI scanner.

In Example 9, at least one of the plurality of scanner field sensors of any one or more of Examples 1-8 are optionally configured to detect an active scan of the MRI scanner, and wherein the IMD of any one or more of Examples 1-8 is optionally configured to terminate or inhibit antitachycardia pacing (ATP) using the detected active scan.

In Example 10, any one or more of Examples 1-9 optionally include an implantable medical device (IMD) configured to be implanted into a subject, the IMD optionally including a first mode and a magnetic resonance imaging (MRI) mode, a plurality of scanner field sensors configured to receive separate indications of a scanner field, the plurality of scanner field sensors including at least one static magnetic field sensor and at least one active magnetic or electric field sensor, wherein the IMD is optionally configured to receive each of the separate indications of the scanner field, to switch from the first mode to the MRI mode using one or more of the plurality of scanner field sensors, and to switch from the MRI mode to the first mode using each of the plurality of scanner field sensors.

In Example 11, the IMD of any one or more of Examples 1-10 is optionally configured to switch from the first mode to the MRI mode if any one or more of the plurality of scanner field sensors receives an indication of a scanner field.

In Example 12, the IMD of any one or more of Examples 1-11 is optionally configured to switch from the MRI mode to the first mode only if each of the plurality of scanner field sensors receives an indication of no scanner field.

In Example 13, the plurality of scanner field sensors of any one or more of Examples 1-12 optionally includes a first scanner field sensor configured to detect an indication of a magnetic field, the first scanner field sensor including an inductor having a ferromagnetic core and a core saturation detector configured to detect saturation of the inductor and the ferromagnetic core in a strong magnetic field, a second scanner field sensor configured to detect an indication of a magnetic field, the second scanner field sensor including a Hall effect sensor, and optionally a third scanner field sensor configured to detect an indication of a magnetic field or electric field, the third scanner field sensor including an electromagnetic interference (EMI) sensor configured to detect EMI from an active MRI scan.

In Example 14, one or more of Examples 1-13 optionally include receiving each of a plurality of separate indications of a scanner field using a corresponding plurality of scanner field sensors of an IMD, switching an IMD from a first therapy mode to a second therapy mode using one or more of the plurality of scanner field sensors, and switching the IMD from the second therapy mode to the first therapy mode using each of the plurality of scanner field sensors.

In Example 15, the switching the IMD from the first therapy mode to the second therapy mode of any one or more of Examples 1-14 optionally includes if any one or more of the plurality of scanner field sensors receives an indication of a scanner field.

In Example 16, the switching the IMD from the second therapy mode to the first therapy mode of any one or more of Examples 1-15 optionally includes only if each of the plurality of scanner field sensors receives an indication of no scanner field.

In Example 17, the first therapy mode of any one or more of Examples 1-16 optionally includes normal operation of the IMD outside of a scanner field, and wherein the second therapy mode of any one or more of Examples 1-16 optionally includes at least one of a terminated or inhibited shock therapy or a terminated or inhibited antitachycardia pacing (ATP).

In Example 18, the receiving the plurality of separate indicators of a scanner field of any one or more of Examples 1-17 optionally includes receiving information from at least one static magnetic field sensor and at least one active magnetic or electric field sensor, optionally including receiving information from a first scanner field sensor configured to detect an indication of a magnetic field, the first scanner field sensor including an inductor having a ferromagnetic core and a core saturation detector configured to detect saturation of the inductor and the ferromagnetic core in a strong magnetic field, receiving information from a second scanner field sensor configured to detect an indication of a magnetic field, the second magnetic field sensor including a Hall effect sensor, and receiving information from a third scanner field sensor configured to detect an indication of a magnetic or electric field, the third scanner field sensor including an electromagnetic interference (EMI) sensor configured to detect EMI from an active MRI scan.

In Example 19, any one or more of Examples 1-18 optionally include detecting proximity of the IMD to an MRI scanner using at least one of the plurality of scanner field sensors, and terminating or inhibiting shock therapy to the subject using the detected proximity.

In Example 20, any one or more of Examples 1-19 optionally include detecting an active scan of the MRI scanner, and terminating or inhibit antitachycardia pacing (ATP) using the detected active scan.

In Example 21, a system or apparatus can include, or can optionally be combined with any portion or combination of any portions of any one or more of Examples 1-20 to include, means for performing any one or more of the functions of Examples 1-20, or a machine-readable medium including instructions that, when performed by a machine, cause the machine to perform any one or more of the functions of Examples 1-20.

The examples provided herein can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally an example of a system including an IMD configured to be implanted in a subject.

FIG. 2 illustrates generally an example of a system including an IMD implanted in a subject, the IMD wirelessly coupled to an external module.

FIG. 3 illustrates generally an example of a state diagram including a first therapy mode and a second therapy mode.

FIG. 4 illustrates generally an example of a method including changing between a first therapy mode and a second therapy mode.

FIG. 5 illustrates generally an example of a method including terminating or inhibiting shock therapy

FIG. 6 illustrates generally an example of a method including terminating or inhibiting ATP therapy.

### DETAILED DESCRIPTION

Nuclear magnetic resonance (NMR) devices (e.g., an MRI scanner, an NMR spectrometer, or other NMR device) can produce both static and time-varying magnetic and electric fields. For example, an MRI scanner can provide a strong static magnetic field, B₀, such as to align nuclei within a subject to the axis of the B₀ field. The B₀ can provide a slight net magnetization (e.g., a “spin polarization”) among the nuclei in bulk because the spin states of the nuclei are not randomly distributed among the possible spin states. Because the resolution attainable by NMR devices can be related to the magnitude of the B₀ field, a stronger B₀ field can be used to spin polarize the subject's nuclei to obtain finer resolution images. NMR devices can be classified according the magnitude of the B₀ field used during imaging, such as a 1.5 Tesla B₀ field, a 3.0 Tesla B₀ field, etc.

After nuclei are aligned using the B₀ field, one or more radio frequency (RF) magnetic excitation pulses can be delivered such as to alter the alignment of specified nuclei (e.g., within a particular volume or plane to be imaged within the subject). The power, phase, and range of frequencies of the one or more RF excitation pulses can be selected, such as depending on the magnitude of the B₀ field, the type or resonant frequency of the nuclei to be imaged, or one or more other factors. After the RF excitation pulses are turned off, one or more RF receivers can be used to detect a time-varying magnetic field (e.g., a flux) developed by the nuclei as they relax back to a lower energy state, such as the spin polarized state induced by the static magnetic field, B₀.

One or more gradient magnetic fields can also be provided during magnetic resonance (MR), such as to create a slight position-dependent variation in the static polarization field. The variation in the static polarization field slightly alters the resonant frequency of the relaxing nuclei, such as during relaxation after excitation by the one or more RF pulses. Using the gradient field along with the static field can provide “spatial localization” of signals detected by the RF receiver, such as by using frequency discrimination. Using a gradient field can allow a volume or a plane to be imaged more efficiently. In a gradient field example, signals received from relaxing nuclei can include energy in respective unique frequency ranges corresponding to the respective locations of the nuclei.

Active MRI equipment can induce unwanted torques, forces, or heating in an IMD or other conductive implant, or can interfere with operation of the IMD. In certain examples, the interference can include disruption in sensing by the IMD, interference in communication between the IMD and other implants or external modules during MRI operation, or disruption in monitoring or therapeutic function of the IMD.

During an MRI scan, the one or more RF excitation pulses can include energy delivered at frequencies from less than 10 MHz to more than 100 MHz, such as corresponding to the nuclear magnetic resonances of the subject nuclei to be imaged. The gradient magnetic field can include energy delivered at frequencies lower than the RF excitation pulses, because most of the AC energy included in the gradient field is provided when the gradient field is ramping or “slewing.” The one or more gradient magnetic fields can be provided in multiple axes, such as including individual time-varying gradient fields provided in each of the axes to provide imaging in multiple dimensions.

In an example, the static field, B₀, can induce unwanted forces or torques on ferromagnetic materials, such as steel or nickel. The forces or torques can occur even when the materials are not directly within the “bore” of the MRI equipment, because significant fields can exist near the MRI equipment. Moreover, if an electric current is switched on or off in the presence of the B₀ field, a significant torque or force can be suddenly imposed in the plane of the circulation of the current, even though the B₀ field itself is static. The induced force or torque can be minimal for small currents, but the torque can be significant for larger currents, such as those delivered during shock therapy. For example, assuming the circulating current is circulating in a plane normal (e.g., perpendicular) to the static field, the torque can be proportional to the magnitude of the B₀ field, multiplied by the surface area of the current loop, multiplied by the current.

Time-varying fields, such as the gradient field or the field associated with the RF excitation pulse, can present different risks than the static field, B₀. For example, the behavior of a wire loop in the presence of a time-varying magnetic field can be described using Faraday's law, which can be represented by

$$ɛ = - \frac{d {Φ}_{{B}_{1}}}{d t} ,$$

in which ∈ can represent the electromotive force (e.g., in volts), such as developed by a time-varying magnetic flux. The magnetic flux can be represented as

$${Φ}_{B 1} = {\int \int }_{S} {B}_{1} \cdot d S ,$$

in which B₁ can represent an instantaneous magnetic flux density vector (e.g., in Webers per square meter, or Tesla). If B₁ is relatively uniform over the surface S, then the magnetic flux can be approximately Φ_{B1}=|B₁||A|, where A can represent the area of the surface S. Operating MRI equipment can produce a time-varying gradient field having a slew rates in excess of 100 Tesla per second (T/s). The slew rate can be similar to a “slope” of the gradient field, and is thus similar to

$$\frac{d {Φ}_{{B}_{1}}}{d t} .$$

The electromotive force (EMF) of Faraday's law can cause an unwanted heating effect in a conductor, regardless of whether the conductor is ferromagnetic. EMF can induce current flow in a conductor (e.g., a housing of an IMD, one or more other conductive regions within an IMD, or one or more other conductive implants). The induced current can dissipate energy and can oppose the direction of the change of the externally applied field (e.g., given by Lenz's law). The induced current tends to curl away from its initial direction, forming an “eddy current” over the surface of the conductor, such as due to Lorentz forces acting upon electrons moving through the conductor. Because non-ideal conductors have a finite resistivity, the flow of induced current through the conductor can dissipate heat. The induced heat can cause a significant temperature rise in or near the conductor over the duration of the scan. The power dissipated by the eddy current can be proportional to the square of both the peak flux density and the frequency of the excitation.

Generally, induced currents, such as induced by the RF magnetic excitation pulse, can concentrate near the surface of a conductor, a phenomenon that can be referred to as the skin effect. The skin effect can limit both the magnitude and depth of the induced current, thus reducing power dissipation. However, the gradient field can include energy at a much lower frequency than the RF magnetic excitation field, which can more easily penetrate through the housing of the IMD. Unlike the field from the RF excitation pulse, the gradient field can more easily induce bulk eddy currents in one or more conductors within the IMD housing, such as within one or more circuits, capacitors, batteries, or other conductors.

Aside from heating, the EMF can create, among other things, non-physiologic voltages that can cause erroneous sensing of cardiac electrical activity, or the EMF can create a voltage sufficient to depolarize cardiac tissue or render the cardiac tissue refractory, possibly affecting pacing therapy. In an illustrative example, an IMD can be connected to one or more leads, such as one or more subcutaneous or intravascular leads positioned to monitor the patient, or to provide one or more therapies to the patient. In this illustrative example, a surface area of a “circuit” including the lead, the housing of the IMD, and a path through at least partially conductive body tissue between an electrode on the lead and the IMD housing can be more than 300 square centimeters, or more than 0.03 square meters. Thus, using Faraday's law, the EMF developed through the body tissue between the electrode (e.g., a distal tip or ring electrode) of the lead and the housing of the IMD can be more than 3 volts (e.g., more than 0.03 square meters times 100 t/s).

In an MR field, an item, such as an IMD, can be referred to as “MR Safe” if the item poses no known hazard in all MRI environments. In an example, MR Safe items can include non-conducting, non-metallic, non-magnetic items, such as a glass, porcelain, a non-conductive polymer, etc. An item can be referred to as “MR Conditional” in the MR field if the item has been demonstrated to pose no known hazards in a specified MRI environment with specified conditions of use (e.g., static magnetic field strength, spatial gradient, time-varying magnetic fields, RF fields, etc.). In certain examples, MR Conditional items can be labeled with testing results sufficient to characterize item behavior in a specified MRI environment. Testing can include, among other things, magnetically induced displacement or torque, heating, induced current or voltage, or one or more other factors. An item known to pose hazards in all MRI environments, such as a ferromagnetic scissors, can be referred to as “MR Unsafe.”

In an example, an IMD configured to be implanted in a subject can be designed to provide sensing or therapy while a subject is inside of or in close proximity to an MRI scanner. IMDs (e.g., cardiac rhythm management (CRM) systems, etc.) generally have no control over the timing of MRI active scanning or the timing of scanner bore entry or exit. Accordingly, there exists the potential for a subject to receive a shock or other electrostimulation energy just prior to entry into an MRI scanner, or an MRI scan sequence to be initiated while antitachycardia pacing (ATP) is active and the subject is already in the bore. Each of the events can be detrimental to the patent if therapy is allowed to continue in or near the MRI scanner.

In an example, a patient receiving shock therapy in or near the bore of the MRI scanner can be subject to significant forces induced on an implantable lead or other current carrying conductor. For example, sixteen amps in a large magnetic field (e.g., a B₀ field) can generate enough force to provide lead dislodgement. Further, in certain examples, the IMD (e.g., a pulse generator (PG), etc.) can be unable to fully charge shock capacitors of the IMD because large static magnetic fields can saturate the power supply, rendering the IMD incapable of full performance.

In other examples, time-varying gradient magnetic fields or RF magnetic excitation pulses generated during an MRI scan can cause ATP therapy to be disrupted, distorted, or terminated to non-physiologic inputs into the IMD. In certain examples, non-deterministic ATP therapy can accelerate an arrhythmia to ventricular fibrillation (VF). Further, because shock therapy is not available in the bore of an MRI scanner, if allowed to operate normally, an IMD can cause a worsening of an arrhythmia without the availability of shock therapy.

The present inventors have recognized, among other things, that terminating any therapy commanded by the IMD can be the safest approach in or near the bore of an MRI scanner. In an example, therapy termination can require knowledge of proximity of the IMD to the bore of the MRI scanner and knowledge of the presence of active scanning (e.g., a time-varying gradient magnetic field, an RF magnetic excitation pulse, or other active portions of an MRI scan). In an example, therapy delivery (e.g., shock, ATP, etc.) can be terminated when scanner proximity to or active scanning is detected, or when an indication of either is received.

FIG. 1 illustrates generally an example of a system 100 including an IMD 105 configured to be implanted in a subject, the IMD 105 including a first scanner field sensor 106, a second scanner field sensor 107, and a third scanner field sensor 108. In other examples, the IMD 105 can include a different number of scanner field sensors (e.g., more than two, four, etc.).

In an example, the IMD 105 can be configured to provide one or more therapies to the subject (e.g., pacing therapy, defibrillation therapy, etc.), and in certain examples, according to specific therapy modes (e.g., parameters, etc.). The IMD 105 can be configured to receive an indication of proximity to a scanner field, and can be configured to alter at least one therapy parameter in response to the received indication of proximity to the scanner field.

The example of FIG. 1 can include a plurality of scanner field sensors (e.g., the first, second, or third scanner field sensors 106, 107, 108, etc.). In an example, the plurality of scanner field sensors can include a static magnetic field sensor (e.g., a Hall effect sensor, a magnetometer, a reed switch, etc.) configured to detect a static magnetic field, such as the B₀ field, etc., an active magnetic field sensor (e.g., a transducer, an antenna, an inductor, etc.) configured to detect an active magnetic field, such as a time-varying gradient magnetic field, an RF magnetic excitation pulse, or one or more other components of the active magnetic field, or a static or active (e.g., time varying) electric field sensor. In certain examples, any one or more of the plurality of scanner field sensors 106, 107, 108 or other scanner field sensor included herein can optionally be configured to detect or receive an indication of a scanner field, including at least one of an indication of a static or active magnetic field, an indication of a static or active electric field, or an indication of one or more other scanner fields.

The present inventors have recognized, among other things, that by increasing the number of scanner field sensors being used, and by using different types of scanner field sensors, that the sensitivity of detection can be increased. Further, in certain examples, by allowing any one or more scanner field sensor to suspend potentially harmful activity, but requiring more to re-allow the potentially harmful activity, the present inventors have realized, among other things, a system and method to increase subject safety.

In an example, the first scanner field sensor 106 can include an inductor having a ferromagnetic core and a core saturation detector configured to detect saturation of the inductor and the ferromagnetic core. One specific example of an inductor configured to detect a magnetic or scanner field is described in the commonly assigned Stessman U.S. Pat. No. 7,509,167, entitled “MRI DETECTOR FOR IMPLANTABLE MEDICAL DEVICE,” filed on Feb. 16, 2006, which is hereby incorporated by reference in its entirety. In certain examples, saturation of the inductor can be indicative of the presence of a scanner field. In an example, the second scanner field sensor 107 can include a Hall effect sensor (e.g., a multi-axis Hall effect sensor, etc.), such as that disclosed in the commonly assigned, co-pending Maile et al. U.S. Patent Application Ser. No. 61/301,428, entitled “MRI SENSOR BASED ON THE HALL EFFECT FOR CRM IMD APPLICATIONS,” filed on Feb. 4, 2010, which is hereby incorporated by reference in its entirety. In an example, the third scanner field sensor 108 can include an active scan sensor, such as an electromagnetic interference (EMI) sensor configured to detect EMI from an active MRI scan, or one or more other scanner field sensors.

In certain examples, thresholds can be set for each of the plurality of scanner field sensors, such that one or more of the sensors can detect the scanner field as the subject approaches the MRI scanner. In an example, different scanners, manufacturers, etc. can all be associated with different fields about the MRI scanner. In an example, a subject having their foot scanned may not need to have their IMD functionality altered. In other examples, certain IMDs may require altered functions at much farther distances, depending on the design of the IMD, the strength of the scanner field, etc.

In other examples, the IMD 105 can be configured to receive at least one of a user indication of the proximity to the scanner field, or information from an MRI scanner or other device indicative of proximity to the scanner field or of an active MRI scan.

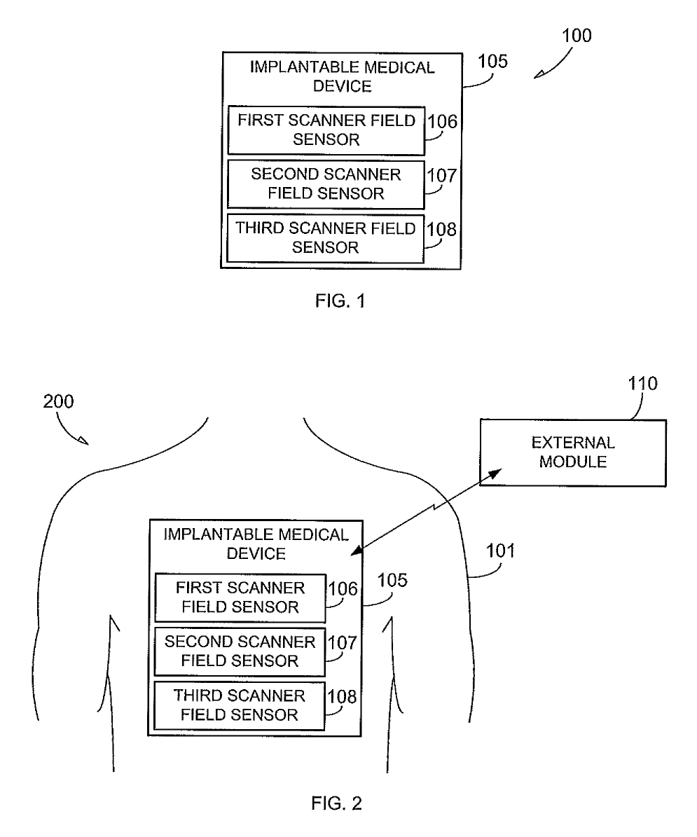

FIG. 2 illustrates generally an example of a system 200 including an IMD 105 including a first scanner field sensor 106, a second scanner field sensor 107, and a third scanner field sensor 108, the IMD 105 implanted in a subject 101, the IMD 105 wirelessly coupled to an external module 110 (e.g., a local or remote programmer). In an example, the IMD 105 can be coupled (e.g., wirelessly, optically, etc.) to one of a local programmer, a remote programmer, or one or more other machines (e.g., an MRI scanner, etc.). In an example, the external module 110 can be configured to receive user information, including MRI instructions.

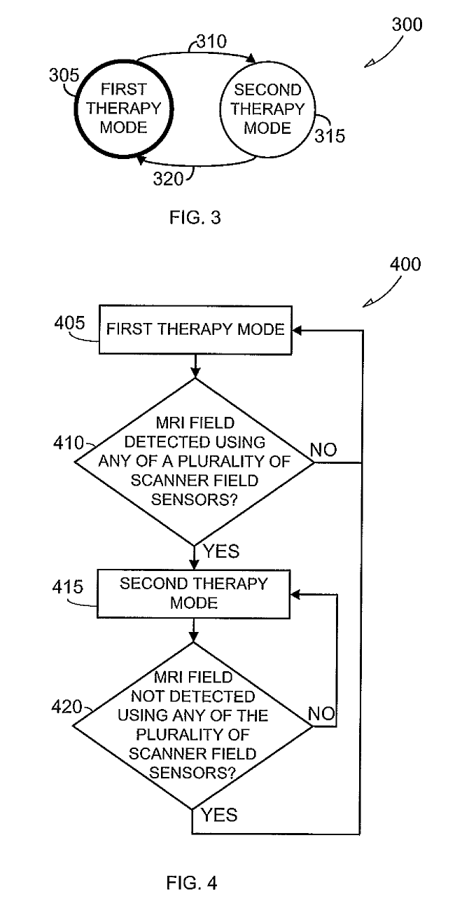

FIG. 3 illustrates generally an example of a state diagram 300 including a first therapy mode 305 and a second therapy mode 315. In an example, the first therapy mode 305 can include normal IMD (e.g., PG, CRM, etc.) operation, such as normal operation outside of a scanner field. In certain examples, the second therapy mode 315 can include an IMD operation having one or more changes to improve or ensure patient safety in an MRI environment. For example, defibrillation shock therapy or ATP can be terminated or inhibited in the presence of a static scanner field.

In an example, at 310, if any one or more of the plurality of scanner field sensors indicate that an MRI scanner is present, or otherwise indicate that a static or time-varying scanner field associated with an NMR device is present, then the second therapy mode 315 is activated. In an example, at 320, if all of the plurality of scanner field sensors indicate that an MRI scanner is not present, or otherwise indicate that the static or time-varying scanner field associated with the NMR device is not present, then the first therapy mode can be reactivated. In other examples, other combinations of scanner field sensors can be used at 310 or 320.

FIG. 4 illustrates generally an example of a method 400 including changing between a first therapy mode and a second therapy mode.

At 405, a first therapy mode is active. In certain examples, the first therapy mode can include a normal operation for an IMD. In other examples, the first therapy mode simply includes one or more of ATP or shock therapy.

At 410, if an MRI field is detected using any one or more of a plurality of scanner field sensors, then a second therapy mode is active at 415. At 410, if the MRI field is not detected using any one or more of the plurality of scanner field sensors, then process flow returns to 405, and a first therapy is active.

At 415, a second therapy mode is active. In certain examples, the second therapy mode 415 can include an IMD operation having one or more changes to improve or ensure patient safety in an MRI environment. For example, defibrillation shock therapy or ATP can be terminated or inhibited in the presence of a static or active scanner field.

At 420, if an MRI field is not detected using any of the plurality of scanner field sensors, then process flow returns to the first therapy mode at 405. At 420, if the MRI field is still detected using any one or more of the plurality of scanner field sensors, then process flow can return to the second therapy mode at 415.

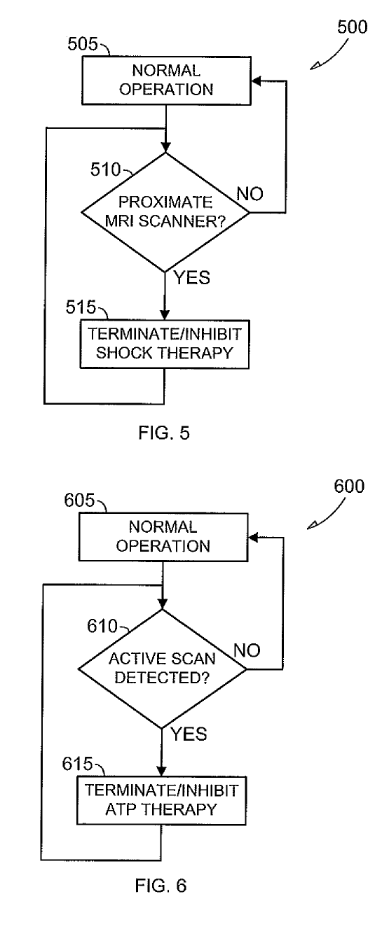

FIG. 5 illustrates generally an example of a method 500 including terminating or inhibiting shock therapy (e.g., a defibrillation shock, etc.).

At 505, normal operation is active. At 510, if an IMD is proximate an MRI scanner, then shock therapy can be terminated or inhibited at 515. At 510, if the IMD is not proximate an MRI scanner, then process flow can return to normal operation at 505. In an example, a scanner field sensor can be used to determine if the IMD is proximate the MRI scanner.

FIG. 6 illustrates generally an example of a method 600 including terminating or inhibiting ATP therapy.

At 605, normal operation is active. At 610, if an IMD detects an active scan, then ATP therapy can be terminated or inhibited at 615. At 610, if the IMD does not detect an active scan, then process flow can return to normal operation at 605. In an example, the IMD can detect an active scan using a scanner field sensor.

Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile tangible computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
an implantable medical device (IMD) configured to be implanted into a subject, the IMD including a first therapy mode and a second therapy mode, the second therapy mode different than the first therapy mode;
a plurality of scanner field sensors configured to receive separate indications of a scanner field; and
wherein the IMD is configured to receive each of the separate indications of the scanner field, to switch from the first therapy mode to the second therapy mode using one or more of the plurality of scanner field sensors, and to switch from the second therapy mode to the first therapy mode using each of the plurality of scanner field sensors.

2. The system of claim 1, wherein the scanner field includes a static magnetic or time-varying magnetic or electric field associated with a nuclear magnetic resonance (NMR) device.

3. The system of claim 1, wherein the IMD is configured to switch from the first therapy mode to the second therapy mode if any one or more of the plurality of scanner field sensors receives an indication of a scanner field.

4. The system of claim 3, wherein the IMD is configured to switch from the second therapy mode to the first therapy mode only if each of the plurality of scanner field sensors receives an indication of no scanner field.

5. The system of claim 1, wherein the first therapy mode includes normal operation of the IMD, and wherein the second therapy mode includes at least one of a terminated or inhibited shock therapy or a terminated or inhibited antitachycardia pacing (ATP).

6. The system of claim 1, wherein the plurality of scanner field sensors includes at least one static magnetic or electric field sensor and at least one active magnetic or electric field sensor.

7. The system of claim 6, wherein the plurality of scanner field sensors includes:
a first scanner field sensor configured to detect an indication of a magnetic field, the first scanner field sensor including an inductor having a ferromagnetic core and a core saturation detector configured to detect saturation of the inductor and the ferromagnetic core in a strong magnetic field;
a second scanner field sensor configured to detect an indication of a magnetic field, the second scanner field sensor including a Hall effect sensor; and
a third scanner field sensor configured to detect an indication of a magnetic or electric field, the third scanner field sensor including an electromagnetic interference (EMI) sensor configured to detect EMI from an active magnetic resonance imaging (MRI) scan.

8. The system of claim 1, wherein at least one of the plurality of scanner field sensors are configured to detect a proximity to an MRI scanner; and
wherein the IMD is configured to terminate or inhibit shock therapy using the detected proximity to the MRI scanner.

9. The system of claim 1, wherein at least one of the plurality of scanner field sensors are configured to detect an active scan of the MRI scanner; and
wherein the IMD is configured to terminate or inhibit antitachycardia pacing (ATP) using the detected active scan.

10. A system comprising:
an implantable medical device (IMD) configured to be implanted into a subject, the IMD including a first mode and a magnetic resonance imaging (MRI) mode;
a plurality of scanner field sensors configured to receive separate indications of a scanner field, the plurality of scanner field sensors including at least one static magnetic field sensor and at least one active magnetic or electric field sensor; and
wherein the IMD is configured to receive each of the separate indications of the scanner field, to switch from the first mode to the MRI mode using one or more of the plurality of scanner field sensors, and to switch from the MRI mode to the first mode using each of the plurality of scanner field sensors.

11. The system of claim 10, wherein the IMD is configured to switch from the first mode to the MRI mode if any one or more of the plurality of scanner field sensors receives an indication of a scanner field.

12. The system of claim 10, wherein the IMD is configured to switch from the MRI mode to the first mode only if each of the plurality of scanner field sensors receives an indication of no scanner field.

13. The system of claim 10, wherein the plurality of scanner field sensors includes:
a first scanner field sensor configured to detect an indication of a magnetic field, the first scanner field sensor including an inductor having a ferromagnetic core and a core saturation detector configured to detect saturation of the inductor and the ferromagnetic core in a strong magnetic field;
a second scanner field sensor configured to detect an indication of a magnetic field, the second scanner field sensor including a Hall effect sensor; and
a third scanner field sensor configured to detect an indication of a magnetic or electric field, the third scanner field sensor including an electromagnetic interference (EMI) sensor configured to detect EMI from an active MRI scan.

14. A method including:
receiving each of a plurality of separate indications of a scanner field using a corresponding plurality of scanner field sensors of an IMD;
switching an IMD from a first therapy mode to a second therapy mode using one or more of the plurality of scanner field sensors; and
switching the IMD from the second therapy mode to the first therapy mode using each of the plurality of scanner field sensors.

15. The method of claim 14, wherein the switching the IMD from the first therapy mode to the second therapy mode includes if any one or more of the plurality of scanner field sensors receives an indication of a scanner field.

16. The method of claim 15, wherein the switching the IMD from the second therapy mode to the first therapy mode includes only if each of the plurality of scanner field sensors receives an indication of no scanner field.

17. The method of claim 14, wherein the first therapy mode includes normal operation of the IMD outside of a scanner field, and wherein the second therapy mode includes at least one of a terminated or inhibited shock therapy or a terminated or inhibited antitachycardia pacing (ATP).

18. The method of claim 14, wherein the receiving the plurality of separate indicators of a scanner field includes receiving information from at least one static magnetic field sensor and at least one active magnetic or electric field sensor, including:
receiving information from a first scanner field sensor configured to detect an indication of a magnetic field, the first scanner field sensor including an inductor having a ferromagnetic core and a core saturation detector configured to detect saturation of the inductor and the ferromagnetic core in a strong magnetic field;
receiving information from a second scanner field sensor configured to detect an indication of a magnetic field, the second magnetic field sensor including a Hall effect sensor; and
receiving information from a third scanner field sensor configured to detect an indication of a magnetic or electric field, the third scanner field sensor including an electromagnetic interference (EMI) sensor configured to detect EMI from an active MRI scan.

19. The method of claim 14, including:
detecting a proximity of the IMD to an MRI scanner using at least one of the plurality of scanner field sensors; and
terminating or inhibiting shock therapy to the subject using the detected proximity.

20. The method of claim 14, including:
detecting an active scan of the MRI scanner; and
terminating or inhibit antitachycardia pacing (ATP) using the detected active scan.
